(19) United States
(12) Patent Application Publication
(10) Pub. No.: US 2026/0162277 A1
(43) Pub. Date: Jun. 11, 2026

(54) GAIT BEHAVIOR VISUALIZATION METHOD, PROGRAM, AND DEVICE

(71) Applicants: Hitachi High-Tech Corporation, Tokyo (JP); NATIONAL UNIVERSITY CORPORATION HOKKAIDO UNIVERSITY, Sapporo-shi, Hokkaido (JP)

(72) Inventors: Daisuke FUKUI, Tokyo (JP); Ken KADOYA, Sapporo-shi (JP); Kentaro HOUMAN, Sapporo-shi (JP); Masatoshi MIYAKE, Tokyo (JP); Takeshi TANAKA, Tokyo (JP); Hiromitsu NAKAGAWA, Tokyo (JP)

(73) Assignees: Hitachi High-Tech Corporation, Tokyo (JP); NATIONAL UNIVERSITY CORPORATION HOKKAIDO UNIVERSITY, Sapporo-shi, Hokkaido (JP)

(21) Appl. No.: 18/703,814

(22) PCT Filed: Oct. 6, 2022
(86) PCT No.: PCT/JP2022/037464

§ 371 Date: Apr. 23, 2024

(30) Foreign Application Priority Data
Oct. 28, 2021 (JP) 2021-176847

(51) Int. Cl. G06T 7/215

(52) U.S. Cl. CPC G06T 7/215; G06T 2207/10028; G06T 2207/30241

(57) ABSTRACT

Skeleton information is visualized in response to diverse needs of individual sites. A gait behavior visualization system stores skeleton data, which is time-series data indicating trajectories of a plurality of sites set for a measurement subject in a three-dimensional space for a gait of the measurement subject, period determination data, which is information used for specifying a time corresponding to each of periods of one gait for the skeleton data, and a perspective defined by designating the period, the site, and a displaying method for the trajectory; specifies a time corresponding to the period for the skeleton data by comparing the skeleton data and the period determination data; and generates, based on the skeleton data for the period designated in the perspective, a screen on which the trajectory of the site designated in the perspective is presented according to the displaying method designated in the perspective.

### TECHNICAL FIELD

The present invention relates to a gait behavior visualization method, a program, and a device.

### BACKGROUND ART

The present application is based on and claims priority from JP Application Serial Number 2021-176847, filed Oct. 28, 2021, the disclosure of which is hereby incorporated by reference herein in its entirety.

In recent years, devices capable of acquiring three-dimensional data, such as an optical camera and a depth camera (a depth sensor) have been widely used, and various information provision mechanisms have been proposed that utilize human skeleton information obtained through 3D sensing using such devices.

For example, PTL 1 describes a motion information processing device configured to provide display information that facilitates evaluation of a gait situation. The motion information processing device acquires motion information on a subject who executes a gait motion; generates, based on the motion information, trajectory information indicating a position of a foot landing point of the subject and a movement of the subject; and causes a display unit to display information selected by a selection operation from the trajectory information. The motion information processing device generates, as the trajectory information, trajectory information indicating an angle of a predetermined site on a body of the subject or a movement trajectory of a characteristic position of the subject.

For example, PTL 2 describes a gait behavior display system configured to analyze and display a gait behavior of a pedestrian in an easy-to-understand manner, and to suggest improvement methods, thereby leading to prevention, early detection, and appropriate treatment of locomotive syndrome. The gait behavior display system selects a measurement of the pedestrian and a measurement of a reference pedestrian to be compared with the pedestrian, displays a first gait model in which one gait of the pedestrian is displayed as an animation, displays a second gait model in which one gait of the reference pedestrian is displayed as an animation, and displays a size of predetermined feature data related to the measurement of the pedestrian and a size of predetermined feature data related to the measurement of the reference pedestrian in a comparable manner.

### CITATION LIST

### Patent Literature

PTL 1: JP 2015-42241A

PTL 2: JP 2019-187878A

### SUMMARY OF INVENTION

### Technical Problem

There are various needs for visualization of information based on skeleton information for each site where the information is used. For example, doctors in medical settings desire to visualize skeleton information from a medical viewpoint, and for example, trainers at training gyms desire to visualize skeleton information from a viewpoint of training efficiency, safety, or the like.

In PTL 1, the trajectory information indicating a position of a foot landing point of a subject who executes a gait motion and a movement of the subject is generated, and the information selected by a selection operation from the trajectory information is displayed on the display unit. In PTL 2, one gait of the pedestrian and one gait of the reference pedestrian are displayed as an animation, and the size of predetermined feature data is displayed in a comparable manner. However, in both PTL 1 and PTL 2, the skeleton information is only visualized based on a general viewpoint, and no particular consideration is given to visualizing skeleton information in detail to meet the needs of individual sites.

The invention has been made in view of such a background, and an object of the invention is to provide a gait behavior visualization method, a program, and a device capable of visualizing skeleton information in response to various needs in individual sites.

### Solution to Problem

According to one aspect of the invention for achieving the above object, there is provided a gait behavior visualization method that includes: A step of storing, using an information processing device including a processor and a memory, skeleton data, which is time-series data indicating trajectories of a plurality of sites set for a measurement subject in a three-dimensional space, for a gait of the measurement subject, period determination data, which is information used for specifying a time corresponding to each of periods of one gait, for the skeleton data, and a perspective defined by designating the period, the site, and a displaying method for the trajectory; a step of specifying, using the information processing device, a time corresponding to the period for the skeleton data by comparing the skeleton data and the period determination data; and a step of generating, using the information processing device based on the skeleton data for the period designated in the perspective, a screen on which a trajectory of the site designated in the perspective is presented according to the displaying method designated in the perspective.

Other problems disclosed by the present application and methods for solving the problems will be made clear by the detailed description and drawings.

### Advantageous Effects of Invention

According to the invention, skeleton information can be visualized in response to various needs in individual sites.

### BRIEF DESCRIPTION OF DRAWINGS

FIG. 1 is a diagram showing a schematic configuration of a gait behavior visualization system.

FIG. 2A is an example of skeleton data.

FIG. 2B is an example of a site set as a measurement point.

FIG. 3A is an example of periods (time intervals) of one gait.

FIG. 3B is an example of period determination data.

FIG. 4 is an example of perspective data.

FIG. 5 is an example of feature management data.

FIG. 6 is an example of period specifying data.

FIG. 7 is an example of feature data.

FIG. 8 is an example of an information processing device used in the configuration of the gait behavior visualization system.

FIGS. 9A-9C are an example of information displayed on an information presentation screen, in which 9A, 9B, and 9C are images (or animation videos) showing trajectories of a pelvis of a measurement subject on a frontal plane, a horizontal plane, and a sagittal plane, respectively.

FIGS. 10A-10C are an example of information displayed on the information presentation screen, in which 10A, 10B, and 10C are images (or animation videos) showing trajectories of a line segment connecting a right shoulder and a left shoulder of the measurement subject on the frontal plane, the horizontal plane, and the sagittal plane, respectively.

FIGS. 11A-11C are an example of information displayed on the information presentation screen, in which 11A, 11B, and 11C are images (or animation videos) showing trajectories of the line segment connecting the right shoulder and the left shoulder of the measurement subject on the frontal plane, the horizontal plane, and the sagittal plane, respectively.

FIGS. 12A-12C are an example of information displayed on the information presentation screen, in which 12A, 12B, and 12C are images (or animation videos) showing trajectories of a lower left limb of the measurement subject relative to the pelvis (a reference point) of the measurement subject on the frontal plane, the horizontal plane, and the sagittal plane, respectively.

FIG. 13 is a flowchart showing gait behavior visualization processing.

FIG. 14A is an example of the information presentation screen.

FIG. 14B is an example of the information presentation screen.

FIG. 14C is an example of the information presentation screen.

FIG. 14D is an example of the information presentation screen.

FIG. 14E is an example of the information presentation screen.

FIGS. 14F and 14G are an example of a feature displayed on the information presentation screen.

FIG. 15 is a diagram showing a schematic configuration of a gait behavior visualization system of a second embodiment.

FIG. 16 is a flowchart showing gait behavior visualization processing of the second embodiment.

FIG. 17 is an example of analysis result data.

FIG. 18 is a diagram showing a schematic configuration of a gait behavior visualization system of a third embodiment.

FIG. 19 is a flowchart showing gait behavior visualization processing of the third embodiment.

FIG. 20 is an example of a print image.

### DESCRIPTION OF EMBODIMENTS

Hereinafter, embodiments will be described with reference to the drawings. The following description and drawings are merely examples for describing the invention, and are omitted or simplified as appropriate to clarify the description. The invention can be implemented in various other aspects. Unless otherwise specified, each component may be either a single unit or multiple units.

In the following description, the same or similar components are denoted by the same reference numerals, and a redundant description thereof may be omitted. In the following description, a letter “S” before the reference sign means a processing step. In the following description, various types of information may be described by expressions such as “information”, “data”, and “table”, but various types of information may be handled using data structures other than those illustrated.

### First Embodiment

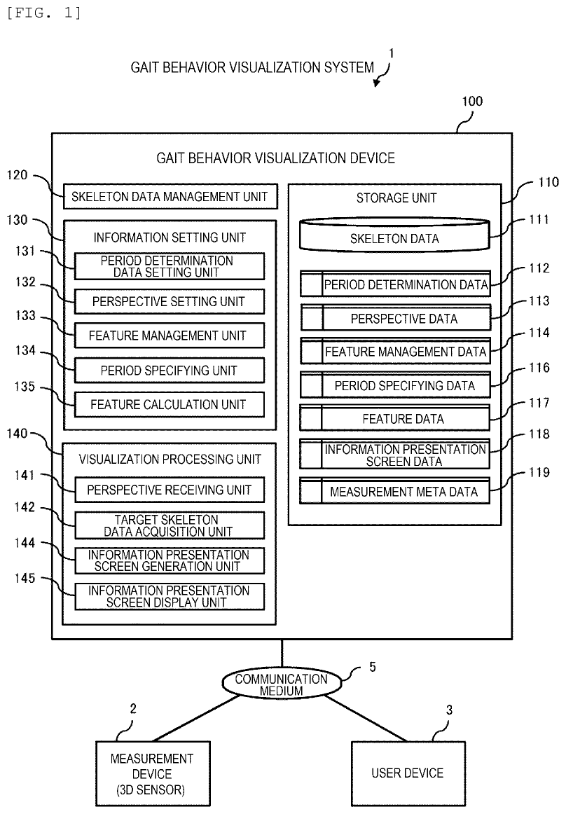

FIG. 1 shows a schematic configuration of an information processing system (hereinafter, referred to as a “gait behavior visualization system 1”) shown as a first embodiment. As shown in FIG. 1, the gait behavior visualization system 1 includes a gait behavior visualization device 100, a measurement device 2, and a user device 3. These devices are all implemented using an information processing device (a computer), and are connected to each other via a communication medium 5 (a communication infrastructure) such that they can communicate bidirectionally. Two or more of the gait behavior visualization device 100, the measurement device 2, and the user device 3 may be implemented as a common information processing device (hardware).

The gait behavior visualization device 100 generates a screen (hereinafter, referred to as an “information presentation screen”) on which information based on a time-series data group (hereinafter, referred to as “skeleton data”) indicating a trajectory (a motion trajectory) of each of measurement points set in a plurality of sites of a body of a measurement subject (subject) in a three-dimensional space, which is generated based on information measured by the measurement device 2, is described. The gait behavior visualization device 100 transmits the generated information presentation screen to the user device 3, and the user device 3 presents the information presentation screen to a user via a user interface.

As shown in FIG. 1, the gait behavior visualization device 100 has functions of a storage unit 110, an information setting unit 130, a skeleton data management unit 120, and a visualization processing unit 140.

The storage unit 110 stores skeleton data 111, period determination data 112, perspective data 113, feature management data 114, period specifying data 116, feature data 117, information presentation screen data 118, and measurement meta data 119.

The skeleton data 111 is a set of skeleton data (a set of data in which a measurement time and position coordinates (X, Y, Z) of each point at the measurement time are associated with each other) for each time cross-section measured at predetermined time intervals for each measurement point of the measurement subject. The skeleton data 111 is generated for each measurement subject and managed in association with an identifier of the measurement subject (hereinafter referred to as a “measurement subject ID”).

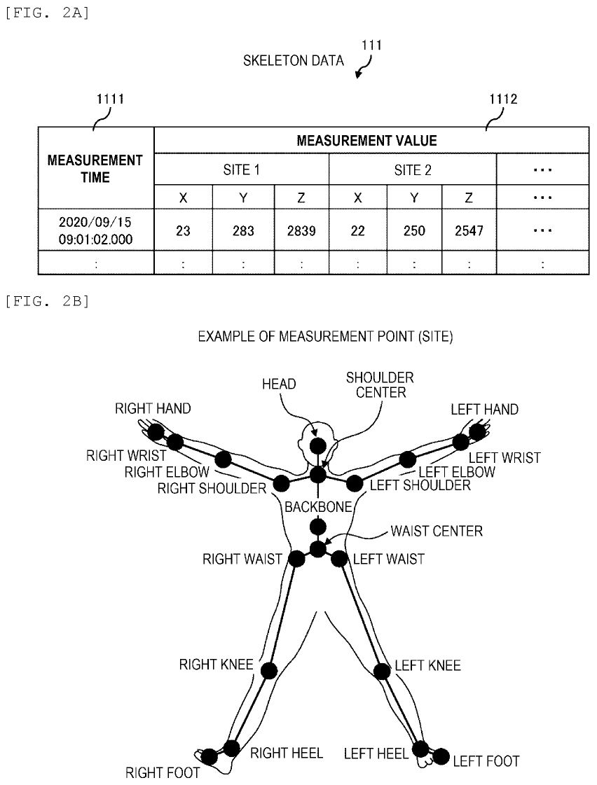

FIG. 2A shows an example of the skeleton data 111. The illustrated skeleton data 111 includes a plurality of entries (records) each including items of a measurement time 1111 and a measurement value 1112. One entry of the skeleton data 111 corresponds to a certain measurement time (time cross-section). Among the above items, a date and time (a time stamp) when the measurement is performed is stored in the measurement time 1111. Coordinate values (X, Y, Z) in the three-dimensional space that are measured for each measurement point (site) are stored in the measurement value 1112.

FIG. 2B shows an example of a site serving as a measurement point. As shown in FIG. 2B, for example, a main portion, an indirect portion, or the like (a head, a left hand, a left wrist, a left elbow, a left shoulder, a shoulder center, a right shoulder, a right elbow, a right wrist, a right hand, a backbone, a left waist, a waist center, a right waist, a left knee, a right knee, a left heel, a right heel, a left foot, a right foot, or the like) of a body of a person is set as the measurement point.

Referring back to FIG. 1, the period determination data 112 includes information (hereinafter, referred to as “period determination data”) used for specifying a time (the measurement time 1111) of the skeleton data 111 that corresponds to each of periods (including a time point (moment)) obtained by dividing one gait of a person into a plurality of time intervals.

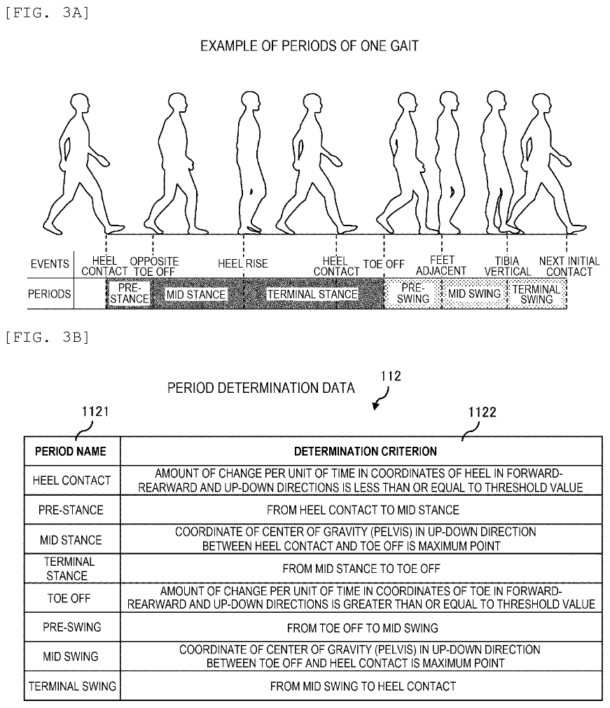

FIG. 3A shows an example of the above periods. In the example in FIG. 3A, one gait of a person is divided into respective periods of “heel contact”, “pre-stance”, “mid stance”, “terminal stance”, “toe off”, “pre-swing”, “mid swing”, and “terminal swing” in chronological order from start of the gait. The method of dividing a single gait is not necessarily limited.

FIG. 3B shows an example of the period determination data 112. As shown in FIG. 3B, the illustrated period determination data 112 includes a plurality of entries (records) each including items of a period name 1121 and a determination criterion 1122. The period determination data 112 is set by the user via a user interface provided by the user device 3, for example.

Among the above items, a name of each period (hereinafter, referred to as a “period name”) is stored in the period name 1121. A determination criterion that is a condition for specifying a time of the period is stored in the determination criterion 1122. Although the illustrated determination criterion is described in a natural language, the determination criterion can be described using, for example, a predetermined programming language when implemented in the gait behavior visualization device 100.

Referring back to FIG. 1, the perspective data 113 includes information (hereinafter, referred to as “perspective data”) in which a perspective when visualizing information based on the skeleton data 111 (a perspective when generating information to be described on the information display screen) is defined. The perspective data is customized for each site where the gait behavior visualization system 1 is introduced, for example. For example, when the use site is a medical site, the perspective data defines a perspective desired by a doctor or the like. For example, when the use site is a training gym, the perspective data defines a perspective desired by a trainer or the like.

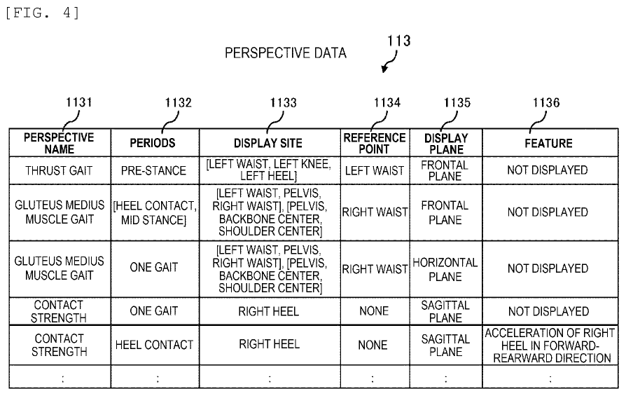

FIG. 4 shows an example of the perspective data 113. The illustrated perspective data 113 includes a plurality of entries (records) each including items of a perspective name 1131, a period 1132, a display site 1133, a reference point 1134, a display plane 1135, and a feature 1136. The perspective data 113 is set by the user via the user interface provided by the user device 3, for example.

Among the above items, a name of a perspective (perspective identification information) is stored in the perspective name 1131.

Information indicating the above-described period to be targeted in the perspective is stored in the period 1132.

A name of a site to be visualized in the perspective (hereinafter, referred to as a “display site”) is stored in the display site 1133. In the display site 1133, a line segment connecting a plurality of sites (a measurement point group constituting the line segment) may be designated. A plurality of display sites enclosed by square brackets “[]” may be stored in the display site 1133, and this means a line segment connecting the plurality of display sites inside the square brackets.

Information indicating a site serving as a reference when visualizing the display site (hereinafter, referred to as a “reference point”) is stored in the reference point 1134. For example, a measurement point with little shake throughout a gait is selected as the reference point. For example, when information indicating a site serving as a reference point is stored in the reference point 1134, information on the display site is visualized as a trajectory of a relative position (position coordinates) with the site as a reference. When “none” is stored in the reference point 1134, information on the display site is visualized using absolute coordinates in the three-dimensional space.

Information for designating a displaying method for a trajectory based on the skeleton data 111 (a frontal plane, a horizontal plane, and a sagittal plane, hereinafter, collectively referred to as a “display plane”) is stored in the display plane 1135.

A feature to be displayed on an information display screen generated according to the perspective is stored in the feature 1136. When the feature is not displayed on the information display screen, “not displayed” is stored in the feature 1136.

Returning to FIG. 1, the feature management data 114 includes information (hereinafter, referred to as “feature management data”) used when a feature calculation unit 135 calculates a feature based on the skeleton data 111. The feature is, for example, a movement and correlation of joints and axes of a measurement subject during a gait. The feature management data 114 is set by the user via the user interface provided by the user device 3, for example.

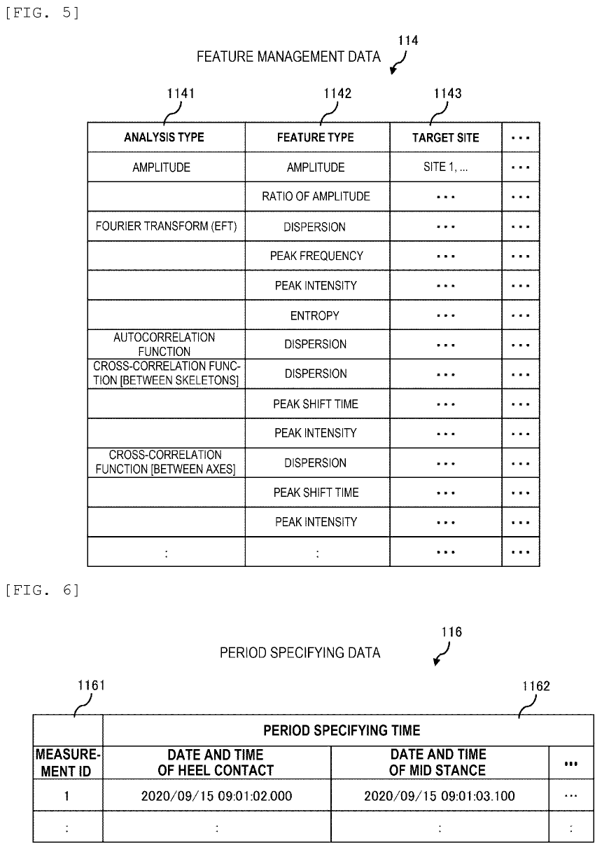

FIG. 5 shows an example of the feature management data 114. In the feature management data 114, information used when the feature calculation unit 135 calculates the feature based on the skeleton data 111 is managed.

As shown in FIG. 5, the feature management data 114 includes a plurality of entries (records) each including items of an analysis type 1141, a feature type 1142, a target site 1143, and the like. The feature management data 114 may further include other pieces of information.

Among the above items, information indicating an analysis method for calculating the feature based on the skeleton data 111 is stored in the analysis type 1141. Information indicating a type of the feature (hereinafter, referred to as a “feature type”) is stored in the feature type 1142. Information indicating a site related to calculation of a feature (hereinafter, referred to as a “target site”) is stored in the target site 1143.

Referring back to FIG. 1, the period specifying data 116 includes a time (a start time, an end time, a time point, and the like of a period, hereinafter, referred to as a “period specifying time”) corresponding to each period in the skeleton data 111 and specified by a period specifying unit 121 of the information setting unit 130, which is to be described later.

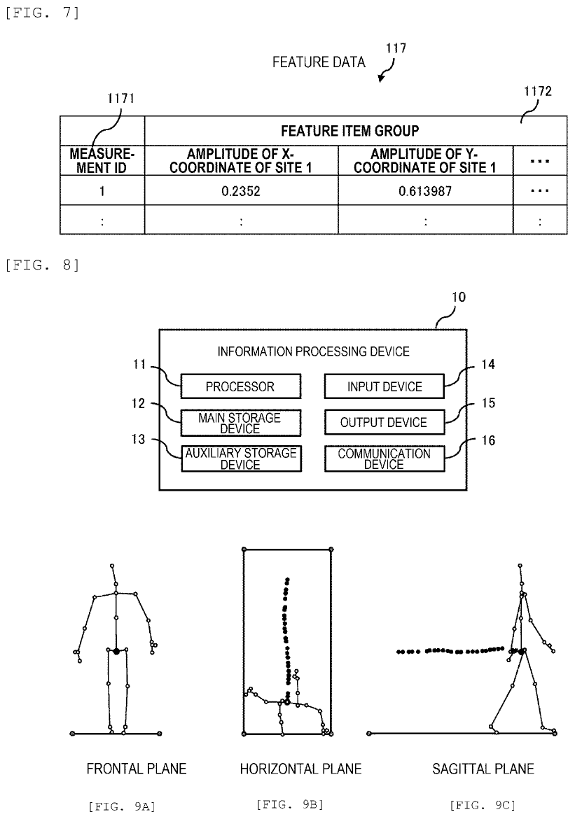

FIG. 6 shows an example of the period specifying data 116. As shown in FIG. 6, the illustrated period specifying data 116 includes one or more entries (records) each including items of a measurement ID 1161 and a period specifying time 1162. One entry of the period specifying data 116 corresponds to one measurement opportunity for one gait of the measurement subject.

Among the above items, an identifier (hereinafter, referred to as a “measurement ID”) assigned for each measurement opportunity is stored in the measurement ID 1161. The above-described period specifying time is stored in the period specifying time 1162.

Returning to FIG. 1, the feature data 117 includes a feature (a specific value of the feature) calculated by the feature calculation unit 135 of the visualization processing unit 140, which is to be described later, based on the skeleton data 111 and the feature management data 114.

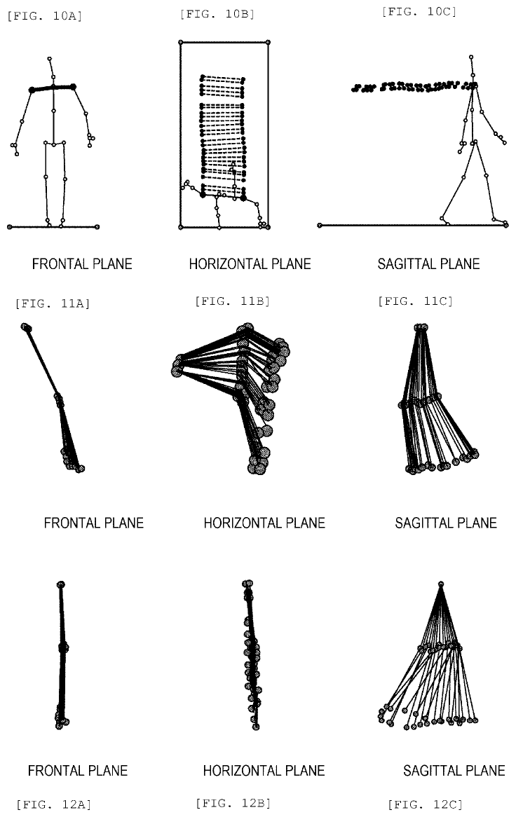

FIG. 7 shows an example of the feature data 117. As shown in FIG. 7, the feature data 117 includes a plurality of entries (records) each including items of a measurement ID 1171 and a feature item group 1172.

Among the above items, the measurement ID is stored in the measurement ID 1171. One or more features calculated by the feature calculation unit 135 based on the skeleton data 111 are stored in the feature item group 1172.

Returning to FIG. 1, the information presentation screen data 118 is screen data (hereinafter, referred to as “information presentation screen data”) generated by an information presentation screen generation unit 144 of the visualization processing unit 140, which is to be described later. The information presentation screen data is, for example, data in a predetermined image data format or data written in a web page description language such as hypertext markup language (HTML). The gait behavior visualization device 100 transmits the generated information presentation screen data 118 to the user device 3. The user device 3 generates an information presentation screen based on the information presentation screen data 118 transmitted from the gait behavior visualization device 100 and presents the information presentation screen to the user.

The measurement meta data 119 includes information (hereinafter, referred to as “measurement meta data”) in which a measurement ID, a measurement subject ID, and information indicating a location of skeleton data (for example, a file name) are associated with one another. The gait behavior visualization device 100 can take correspondence with at least two of the measurement subject ID, the measurement ID, and the skeleton data 111 based on the measurement meta data 119.

The skeleton data management unit 120 shown in FIG. 1 acquires the measurement value and the measurement meta data that are transmitted from the measurement device 2. The skeleton data management unit 120 generates the skeleton data 111 based on the acquired measurement value. The skeleton data management unit 120 manages the acquired measurement meta data as the measurement meta data 119.

The information setting unit 130 shown in FIG. 1 performs processing related to setting of various types of information (the period determination data 112, the perspective data 113, and the feature management data 114) used when visualizing the skeleton data 111.

As shown in FIG. 1, the information setting unit 130 includes a period determination data setting unit 131, a perspective setting unit 132, a feature management unit 133, a period specifying unit 134, and the feature calculation unit 135.

The period determination data setting unit 131 performs processing related to setting of the period determination data 112. The information setting unit 130 receives the setting of the period determination data from the user via the user interface provided by the user device 3, and reflects the received content in the period determination data 112.

The perspective setting unit 132 performs processing related to setting of the perspective data 113. The perspective setting unit 132 receives the setting of the perspective data from the user via the user interface provided by the user device 3, and reflects the received content in the perspective data 113.

The feature management unit 133 manages the feature management data 114. The feature management unit 133 receives the setting of the feature management data from the user via the user interface provided by the user device 3, and reflects the received content in the feature management data 114.

The period specifying unit 134 specifies the above-described period specifying time by comparing the measurement time 1111 in the skeleton data 111 with the determination criterion 1122 in the period determination data 112, and reflects the specified period specifying time in the period specifying data 116.

The feature calculation unit 135 calculates a feature based on the skeleton data 111 and the feature management data 114, and reflects the calculated feature in the feature data 117. Specifically, the feature calculation unit 135 extracts, from the skeleton data 111, skeleton data for a period corresponding to each period (the pre-stance, the terminal stance, and the like) in the period determination data 112 in addition to all periods of one gait, and calculates the feature based on the extracted skeleton data and the feature management data 114. The feature data 117 includes, for example, “amplitude of X coordinate of site 1 in pre-stance”. When calculating the feature, the feature calculation unit 135 may perform preprocessing such as normalization of distance, time, or the like, or smoothing for the purpose of improving calculation accuracy or the like.

The visualization processing unit 140 shown in FIG. 1 receives a designation of a perspective from the user via the user interface provided by the user device 3, generates an information presentation screen based on the received perspective, and transmits the generated information presentation screen to the user device 3.

As shown in FIG. 1, the visualization processing unit 140 includes a perspective receiving unit 141, a target skeleton data acquisition unit 142, the information presentation screen generation unit 144, and an information presentation screen display unit 145.

The perspective receiving unit 141 receives a designation of a perspective from the user via the user interface provided by the user device 3.

The target skeleton data acquisition unit 142 acquires, from the skeleton data 111, skeleton data corresponding to the period 1132 of the perspective received by the perspective receiving unit 141 from the user. Specifically, the target skeleton data acquisition unit 142 acquires, from the perspective data 113, the period 1132 and the display site 1133 of the received perspective, and acquires skeleton data corresponding to the acquired period 1132 and display site 1133 from the skeleton data 111.

The information presentation screen generation unit 144 generates an information presentation screen based on the perspective received by the perspective receiving unit 141 using the skeleton data acquired by the target skeleton data acquisition unit 142. When describing a feature on the information presentation screen, the information presentation screen generation unit 144 acquires a necessary feature from the feature data 117 and describes the feature on the information presentation screen.

The information presentation screen display unit 145 transmits the information presentation screen generated by the information presentation screen generation unit 144 to the user device 3.

The measurement device 2 shown in FIG. 1 includes a three-dimensional sensing device (hereinafter, referred to as a “3D sensor”). In order to capture a gait behavior of the measurement subject, the measurement device 2 measures a movement (a movement trajectory) of each measurement point of a body of the measurement subject during a gait in the three-dimensional space, and transmits the measurement value and the measurement meta data to the gait behavior visualization device 100. The 3D sensor acquires, for example, a distance image (a depth image, range image data) which is data including three-dimensional position information (distance information (depth information)), a distance signal, and the like) for a subject (an object). The 3D sensor is not necessarily limited to those that acquire distance images. For example, a sensor such as an acceleration sensor, an angle sensor, or a gyro sensor may be used as the 3D sensor. Examples of the 3D sensor include a depth sensor such as Kinect (registered trademark), a time of flight (TOF) camera, a stereo camera, a laser imaging detection and ranging (LiDAR), a millimeter wave radar, and an ultrasonic sensor. The 3D sensor may be implemented by combining a plurality of different types of devices.

The user device 3 shown in FIG. 1 cooperates with the information setting unit 130 of the gait behavior visualization device 100, and performs setting (registration, editing, deletion, and the like) of various types of information stored in the storage unit 110 by dialogue processing with the user via the user interface. The user device 3 cooperates with the visualization processing unit 140 of the gait behavior visualization device 100, receives the information presentation screen transmitted from the visualization processing unit 140, and presents (displays, or audio outputs) the information presentation screen to the user via the user interface. The user interface for setting various types of information and presenting the information presentation screen may be generated by the user device 3 or may be provided from the gait behavior visualization device 100.

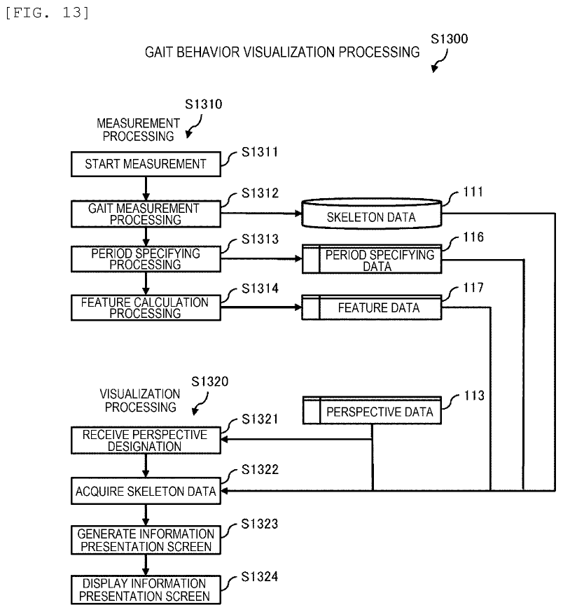

FIG. 8 shows an example of a hardware configuration of an information processing device used for implementing the gait behavior visualization device 100, the measurement device 2, and the user device 3. The illustrated information processing device 10 includes a processor 11, a main storage device 12, an auxiliary storage device 13, an input device 14, an output device 15, and a communication device 16. The information processing device 10 is, for example, a personal computer, a server device, a smartphone, or a tablet.

The information processing device 10 may be implemented, in whole or in part, using a virtual information processing resource provided using a virtualization technique, a process space Separation technique, or the like, such as a virtual server provided by a cloud system. a some of functions provided by the information processing device 10 may be implemented by, for example, a service provided by a cloud system via an application programming interface (API) or the like.

All or a some of the functions provided by the information processing device 10 may be implemented by using, for example, a software as a service (SaaS), a platform as a service (PaaS), or an infrastructure as a service (IaaS). The gait behavior visualization device 100 may be implemented by using, for example, a plurality of information processing devices 10 communicably connected.

The processor 11 shown in FIG. 1 is implemented using, for example, a central processing unit (CPU), a micro processing unit (MPU), a graphics processing unit (GPU), a field programmable gate array (FPGA), an application specific integrated circuit (ASIC), or an artificial intelligence (AI) chip.

The main storage device 12 is a device that stores programs and data, and is, for example, a read only memory (ROM), a random access memory (RAM), or a non-volatile RAM (NVRAM).

The auxiliary storage device 13 is, for example, a solid state drive (SSD), a hard disk drive, an optical storage device (a compact disc (CD), a digital versatile disc (DVD), or the like), a storage system, an IC card, a reading and writing device of a recording medium such as an SD card or an optical recording medium, or a storage area of a cloud server. Programs and data can be read into the auxiliary storage device 13 via a reading device of a recording medium and the communication device 16. The programs and data stored in the auxiliary storage device 13 are read into the main storage device 12 as needed.

The input device 14 is an interface that receives an input from the outside, and is, for example, a keyboard, a mouse, a touch panel, a card reader, a pen input tablet, or a voice input device.

The output device 15 is an interface that outputs various types of information such as processing progress and processing results. The output device 15 is, for example, a display device (a liquid crystal monitor, a liquid crystal display (LCD), a graphic card, or the like) that visualizes the various types of information, a device (an audio output device (a speaker or the like)) that converts the various types of information into audio, or a device (a printer or the like) that converts the various types of information into characters. For example, the information processing device 10 may input and output information to and from another device via the communication device 16.

The input device 14 and the output device 15 constitute a user interface that implements dialogue processing (receiving of information, presentation of information, and the like) with the user.

The communication device 16 is a device that implements communication with another device. The communication device 16 is a wired or wireless communication interface that implements communication with another device via the communication medium 5, and is, for example, a network interface card (NIC), a wireless communication module, or a USB module. The communication medium 5 provides a wired or wireless communication infrastructure. For example, the communication medium 5 is a serial communication medium conforming to a predetermined standard such as a universal serial bus (USB) or RS-232C, a local area network (LAN), a wide area network (WAN), the Internet, a dedicated line, or various public communication networks (wired or wireless).

For example, an operating system, a file system, a data base management system (DBMS) (a relational database, NOSQL, or the like), a key-value store (KVS), or the like may be introduced into the information processing device 10.

The functions of the gait behavior visualization device 100, the measurement device 2, and the user device 3 are implemented by the processor 11 provided in each device reading and executing a program stored in the main storage device 12, or by hardware (FPGA, ASIC, AI chip, or the like) itself constituting each device.

The gait behavior visualization device 100, the measurement device 2, and the user device 3 store various types of information (data) as, for example, a table of a database or a file managed by a file system.

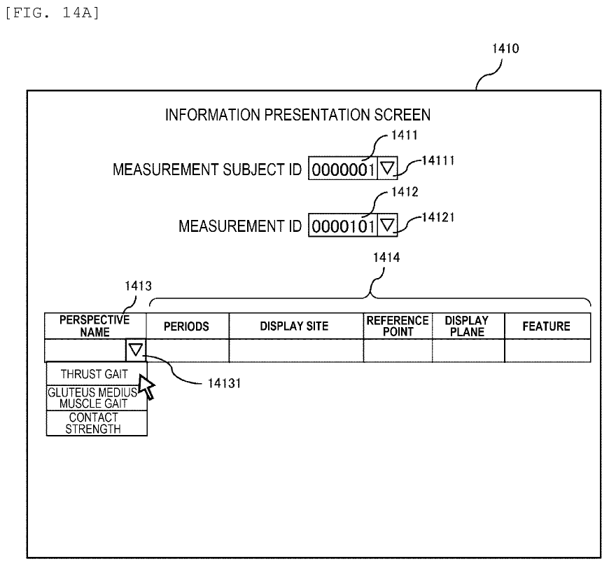

FIG. 9 is an example of information displayed on the information presentation screen, and is an image (or an animation video) indicating a trajectory of a pelvis of the measurement subject, which is generated by the information presentation screen generation unit 144 based on the skeleton data 111. In FIG. 9A, is an image (or an animation video) showing a trajectory of the pelvis of the measurement subject on the frontal plane, FIG. 9B is an image (or an animation video) showing a trajectory of the pelvis of the measurement subject on the horizontal plane, and FIG. 9C is an image (or an animation video) showing a trajectory of the pelvis of the measurement subject on the sagittal plane.

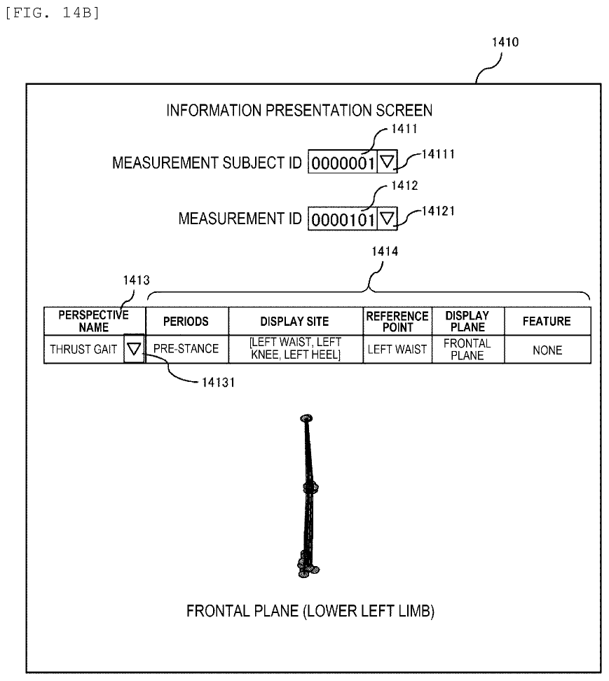

FIG. 10 is another example of the information displayed on the information presentation screen, and shows images (or animation videos) showing trajectories of a line segment connecting a right shoulder and a left shoulder of the measurement subject, which are generated by the information presentation screen generation unit 144 based on the skeleton data 111. In FIG. 10A, is an image (or an animation video) showing a trajectory of the line segment of the measurement subject on the frontal plane, FIG. 10B is an image (or an animation video) showing a trajectory of the line segment of the measurement subject on the horizontal plane, and FIG. 10C is an image (or an animation video) showing a trajectory of the line segment of the measurement subject on the sagittal plane.

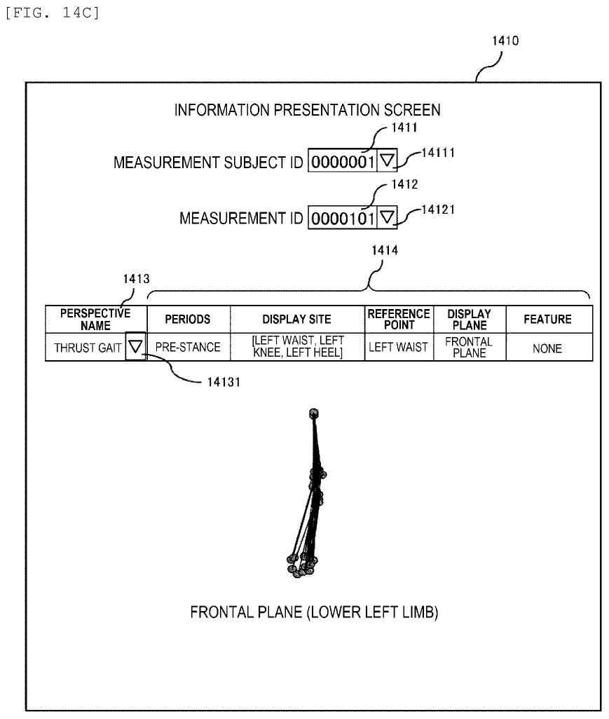

FIG. 11 is still another example of the information described on the information presentation screen, and shows images (or animation videos) showing trajectories of a relative position of an upper left limb with respect to a shoulder center of the measurement subject, which are generated by the information presentation screen generation unit 144 based on the skeleton data 111. In FIG. 11A, is an image (or an animation video) showing a trajectory of the relative position of the upper left limb with respect to the shoulder center (a reference point) of the measurement subject on the frontal plane, FIG. 11B is an image (or an animation video) showing a trajectory of the relative position of the upper left limb with respect to the shoulder center (the reference point) of the measurement subject on the horizontal plane, and FIG. 11C is an image (or an animation video) showing a trajectory of the relative position of the upper left limb with respect to the shoulder center (reference point) of the measurement subject on the sagittal plane.

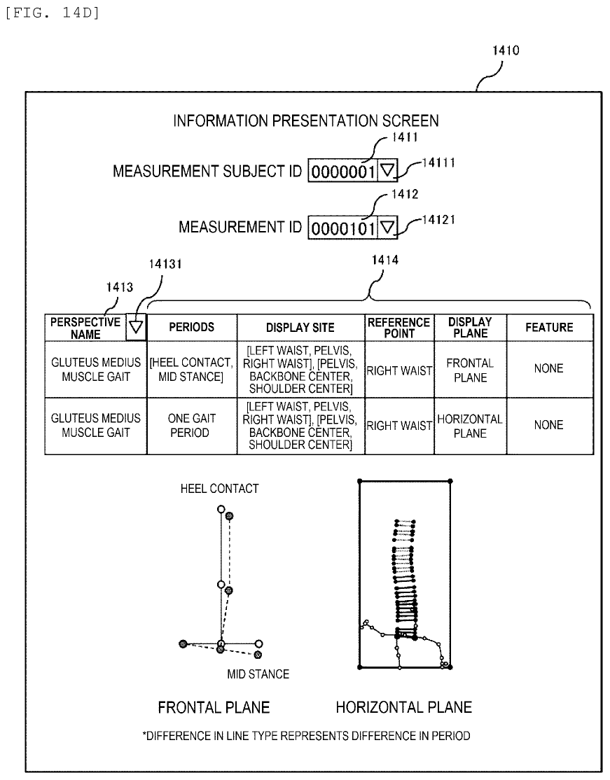

FIG. 12 is yet still another example of the information described on the information presentation screen, and shows images (or animation videos) showing trajectories of a relative position of a lower left limb with respect to the pelvis of the measurement subject, which are generated by the information presentation screen generation unit 144 based on the skeleton data 111. In FIG. 12A, is an image (or an animation video) showing a trajectory of the relative position of the lower left limb with respect to the pelvis (a reference point) of the measurement subject on the frontal plane, FIG. 12B is an image (or an animation video) showing a trajectory of the relative position of the lower left limb with respect to the pelvis (the reference point) of the measurement subject on the horizontal plane, and FIG. 12C is an image (or an animation video) showing a trajectory of the relative position of the lower left limb with respect to the pelvis (the reference point) of the measurement subject on the sagittal plane.

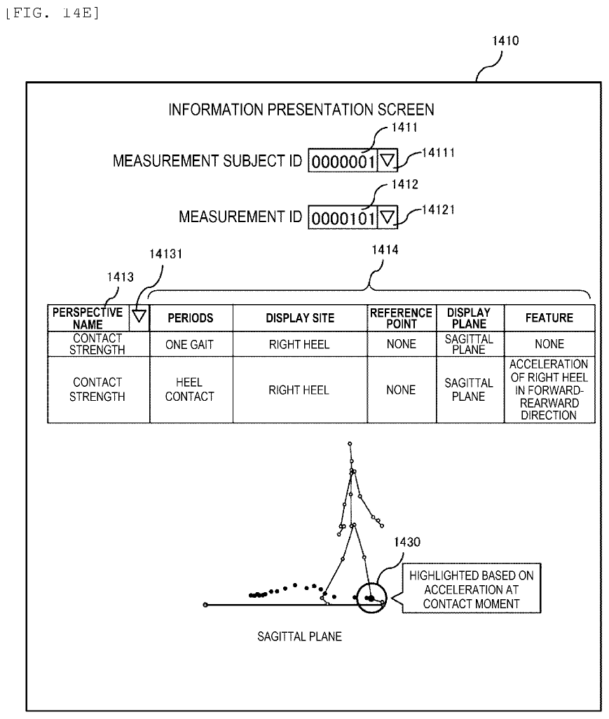

FIG. 13 is a flowchart showing processing performed by the gait behavior visualization device 100 (hereinafter, referred to as “gait behavior visualization processing S1300”). As shown in FIG. 13, the gait behavior visualization processing S1300 includes measurement processing S1310 and visualization processing S1320. Hereinafter, the gait behavior visualization processing S1300 will be described with reference to FIG. 13.

In the measurement processing S1310, the gait behavior visualization device 100 acquires the skeleton data 111, generates the period specifying data 116, and generates the feature data 117.

First, the skeleton data management unit 120 transmits a measurement start instruction to the measurement device 2 (S1311), acquires a measurement value for each measurement point related to a movement of a person during a gait, which is transmitted from the measurement device 2 suitable for the measurement start instruction, and generates the skeleton data 111 (pitch data) including one gait based on the acquired measurement value (S1312). One gait of a person can be defined as, for example, a time interval from when the measurement subject puts a right foot heel on the ground during a gait to when the right foot heel leaves the ground and again comes on the ground. For example, the skeleton data management unit 120 detects “a time when the right foot heel lands” and “a time when the right foot heel leaves the ground and lands again” based on the measurement value transmitted from the measurement device 2, and specifies the times as a start time of one gait and an end time of one gait, respectively.

Subsequently, the period specifying unit 134 of the information setting unit 130 compares the measurement time 1111 in the skeleton data 111 with the determination criterion 1122 in the period determination data 112 to specify the period specifying time of the skeleton data 111 corresponding to each of the periods during a gait of a person, and reflects the specified period specifying time in the period specifying data 116 (S1313).

Subsequently, the feature calculation unit 135 of the information setting unit 130 calculates a feature based on the skeleton data 111, and reflects the calculated feature in the feature data 117 (S1314).

Thus, the measurement processing S1310 ends, and preparation of the skeleton data 111, the period specifying data 116, and the feature data 117 necessary for the visualization processing S1320 is completed.

In the visualization processing S1320, the gait behavior visualization device 100 receives a designation of a perspective from the user, acquires skeleton data used for visualizing the received perspective, generates an information presentation screen based on the specified skeleton data, and transmits the generated information presentation screen to the user device 3 to present the information presentation screen to the user.

First, the perspective receiving unit 141 of the visualization processing unit 140 receives a designation of a perspective from the user via the user interface provided by the user device 3 (S1321).

Subsequently, the target skeleton data acquisition unit 142 of the visualization processing unit 140 acquires the period 1132 and the display site 1133 of the received perspective from the perspective data 113, and acquires skeleton data corresponding to the acquired period 1132 and display site 1133 from the skeleton data 111 (S1322).

Subsequently, the information presentation screen generation unit 144 of the visualization processing unit 140 generates an information presentation screen based on the perspective received by the perspective receiving unit 141 using the skeleton data acquired by the target skeleton data acquisition unit 142. When describing the feature on the information presentation screen, the information presentation screen generation unit 144 acquires a necessary feature from the feature data 117 (S1323).

Subsequently, the information presentation screen display unit 145 of the visualization processing unit 140 transmits the information presentation screen generated by the information presentation screen generation unit 144 to the user device 3. The user device 3 presents the information presentation screen to the user via the user interface (S1324).

Thus, the visualization processing S1320 ends. Thereafter, the gait behavior visualization device 100 may receive a change in perspective from the user and repeatedly execute the visualization processing S1320 based on a newly received perspective.

Next, a specific example of the information presentation screen will be described. In the following example, it is assumed that the perspective data 113 includes the contents shown in FIG. 4.

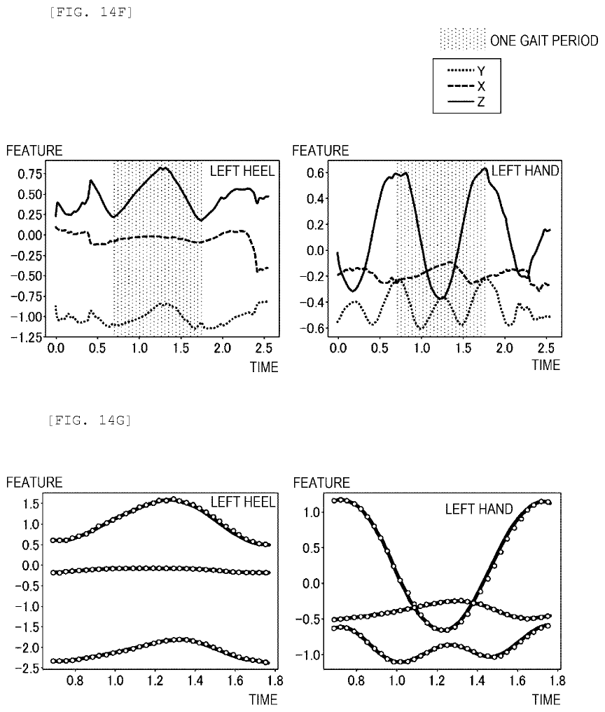

FIG. 14A shows an example of the information presentation screen. The illustrated information presentation screen 1410 includes a measurement subject ID designation field 1411, a measurement ID designation field 1412, a perspective designation field 1413, and a perspective content display field 1414.

The user directly inputs the measurement subject ID into the measurement subject ID designation field 1411 or operates a pull-down menu 14111 to designate the measurement subject ID of the measurement subject whose gait behavior is to be displayed.

Subsequently, the user directly inputs the measurement ID into the measurement ID designation field 1412 or operates a pull-down menu 14121 to designate a target measurement ID. The designation of the measurement ID is not essential, and for example, when the measurement ID is not designated, the measurement ID of the measurement value measured most recently is automatically designated (the same applies to the following examples).

Subsequently, the user directly inputs a perspective in the perspective designation field 1413 or operates a pull-down menu 14131 to designate a perspective. By performing the operation, information (values of the period 1132, the display site 1133, the reference point 1134, the display plane 1135, and the feature 1136) associated with the perspective designated in the perspective designation field 1413 in the perspective data 113 is displayed in the perspective content display field 1414.

FIG. 14B shows a case where the user designates “0000001” in the measurement subject ID 1411, designates “0000101” in the measurement ID designation field 1412, and designates a perspective “thrust gait” in the perspective designation field 1413 in FIG. 14A. Since the perspective “thrust gait” is designated, information associated with the perspective “thrust gait” in the perspective data 113 shown in FIG. 4, that is, a period “pre-stance”, a display site “[left waist, left knee, left heel]”, a reference point “left waist”, a display plane “frontal plane”, and a feature “none” are displayed in the perspective content display field 1414. Below the perspective content display field 1414, trajectories of the display sites “[left hip, left knee, left heel]” (a trajectory of each site of the left waist, left knee, and left heel and a trajectory of a line segment connecting the left waist, left knee, and left heel) viewed from the display plane “frontal plane” in the period “pre-stance” are displayed as images or animation videos. Based the trajectories, the user, who is, for example, a doctor, confirms that a shake in the left knee portion is small, and diagnoses, for example, that the measurement subject is a healthy person.

FIG. 14C shows a case where the user designates “0000002” in the measurement subject ID 1411, designates “0000101” in the measurement ID designation field 1412, and designates a perspective “thrust gait” in the perspective designation field 1413 in FIG. 14A. Since the perspective “thrust gait” is designated as in FIG. 14B, information associated with the perspective “thrust gait” in the perspective data 113 shown in FIG. 4, that is, a period “pre-stance”, a display site “[left waist, left knee, left heel]”, a reference point “left waist”, a display plane “frontal plane”, and a feature “none” are displayed in the perspective content display field 1414. Below the perspective content display field 1414, trajectories of the display sites “[left hip, left knee, left heel]” (a trajectory of each site of the left waist, left knee, and left heel and a trajectory of a line segment connecting the left waist, left knee, and left heel) viewed from the display plane “frontal plane” in the period “pre-stance” are displayed as images or animation videos. Based on the trajectories, the user, who is, for example, a doctor, confirms that a shake in the left knee portion is large, and diagnoses, for example, that the measurement subject is a severe OA patient.

FIG. 14D shows a case where the user designates “0000001” in the measurement subject ID 1411, designates “0000101” in the measurement ID designation field 1412, and designates a perspective “gluteus medius muscle gait” in the perspective designation field 1413 in FIG. 14A. Since the perspective “gluteus medius muscle gait” is designated, information associated with the perspective “gluteus medius muscle gait” in the perspective data 113 shown in FIG. 4 is displayed in the perspective content display field 1414. Here, since there are two rows of information associated with the perspective “gluteus medius muscle gait” in the perspective data 113 shown in FIG. 4, in this case, information of each row, that is, a period “[heel contact, mid stance]”, a display site “[left waist, pelvis, right waist], [pelvis, backbone center, shoulder center]”, a reference point “right waist”, a display plane “frontal plane”, a feature “none” in a first row, and a period “one gait period”, a display site “[left waist, pelvis, right waist], [pelvis, backbone center, shoulder center]”, a reference point “right waist”, a display plane “horizontal plane”, and a feature “none” in a second row are displayed. Below the perspective content display field 1414, a trajectory on the “frontal plane” that corresponds to the first information and a trajectory on the “horizontal plane” that corresponds to the second information are displayed as images or animation videos. In FIG. 14D, line types of line segments connecting the sites represent a difference in period. In this way, when there are a plurality of rows having the same content of the perspective name designation field 1412 in the perspective data 113, information based on each perspective is simultaneously displayed on the information presentation screen. Therefore, the user can simultaneously check information based on related perspectives in a comparable and contrastive manner, and for example, can diagnose a gait behavior from a comprehensive perspective.

FIG. 14E shows a case where the user designates “0000001” in the measurement subject ID 1411, designates “0000101” in the measurement ID designation field 1412, and designates a perspective “contact strength” in the perspective designation field 1413 in FIG. 14A. Since the perspective “contact strength” is designated, information associated with the perspective “contact strength” in the perspective data 113 shown in FIG. 4 is displayed in the perspective content display field 1414. Here, since there are two rows of information associated with the perspective “contact strength” in the perspective data 113 shown in FIG. 4, information of each row, that is, a period “one gait”, a display site “right heel”, a reference point “none”, a display plane “sagittal plane”, a feature “none” in a first row, and a period “heel contact”, a display site “right heel”, a reference point “none”, a display plane “sagittal plane”, and a feature “acceleration of right heel in forward-rearward direction” are displayed. Below the perspective content display field 1414, an image or animation video of a trajectory on the “sagittal plane” that corresponds to the first and second information, and a circle 1430 for highlighting that a feature of the second information “acceleration of right heel in forward-rearward direction” exceeds a predetermined threshold value are displayed in a corresponding “right heel” portion. A display mode of the feature is not limited, and the feature may be displayed in another mode (for example, a color is changed according to a magnitude of the acceleration, or a radius is increased according to the magnitude of the acceleration).

The feature may be displayed in a mode corresponding to each property. For example, as shown in FIG. 14F, a temporal change of the feature may be displayed in a graph. In FIG. 14F, is a graph showing temporal changes of features (displacements) of the left heel and left hand, and FIG. 14G is a graph obtained by smoothing the graph in FIG. 14F. In this way, by visualizing and presenting various features measured for the measurement subject, the user can efficiently acquire various types of information related to the measurement subject.

In the above, although the case where the gait behavior (the trajectories of the site or the line segment connecting the sites) of one measurement subject is displayed on the information presentation screen has been described, for example, gait behaviors of a plurality of measurement subjects may be displayed simultaneously for comparison and contrast. In this case, the gait behaviors may be individually displayed, or may be superimposed and displayed such that a difference can be easily grasped.

As described above, according to the gait behavior visualization system 1 of the present embodiment, the information presentation screen on which the gait behavior is visualized according to the displaying method for the site designated in the perspective and the trajectory can be generated based on the skeleton data for the period of the designated perspective and the information presentation screen can be presented to the user. Therefore, for example, a user who is an expert such as a doctor or a trainer in the site can check the gait behavior from the perspective meeting the needs of the individual site. For example, the user can check a trajectory of a joint point, or a line segment (a pelvis, a line segment connecting a right shoulder and a left shoulder, or the like), and a trajectory of a relative position of the joint point or the line segment with respect to a reference point, or the like on the display plane (the frontal plane, the sagittal plane, or the horizontal plane) suitable for displaying the trajectories. The gait behavior visualization system 1 presents various features to the user in various modes together with the information. Therefore, the user can easily and reliably acquire useful information on the measurement subject, and can effectively use the information for treatment of the measurement subject, advice to the measurement subject, and the like.

### Second Embodiment

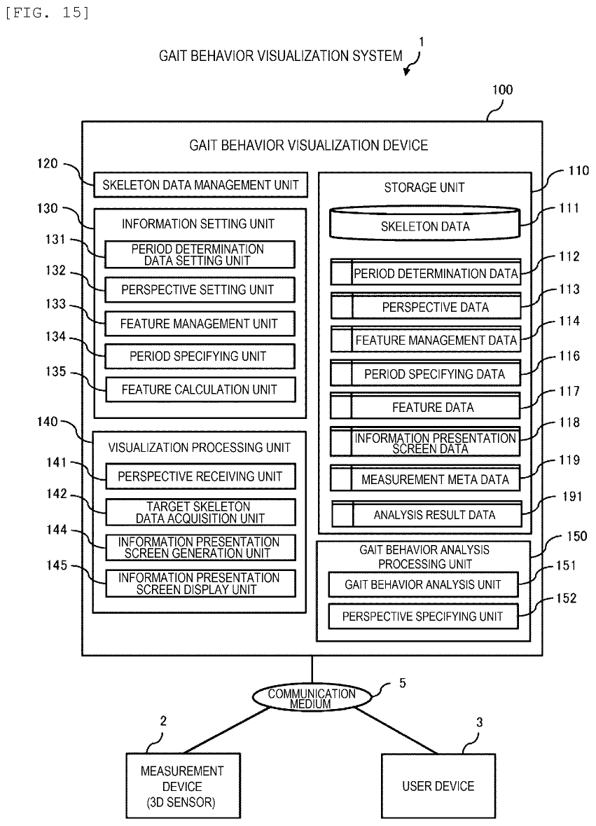

FIG. 15 shows a schematic configuration of the gait behavior visualization system 1 shown as a second embodiment. The gait behavior visualization device 100 in the gait behavior visualization system 1 of the second embodiment further includes a gait behavior analysis processing unit 150 in addition to functions of the gait behavior visualization device 100 of the first embodiment. The storage unit 110 of the gait behavior visualization system 1 of the second embodiment further stores analysis result data 191. The gait behavior visualization device 100 in the gait behavior visualization system 1 of the second embodiment specifies a perspective using a machine learning model trained to analyze a gait of a measurement subject based on a feature.

As shown in FIG. 15, the gait behavior analysis processing unit 150 includes a gait behavior analysis unit 151 and a perspective specifying unit 152. The gait behavior analysis unit 151 analyzes a gait of the measurement subject by inputting the feature data 117 generated by the feature calculation unit 135 to the machine learning model, and generates the analysis result data 191. The perspective specifying unit 152 specifies a perspective based on the analysis result data 191, and inputs the specified perspective to the perspective receiving unit 141 of the visualization processing unit 140.

The machine learning model may perform both analysis of the gait behavior of the measurement subject and specification of the perspective, or may perform only the analysis of the gait behavior of the measurement subject. In the latter case, for example, the feature calculation unit 135 stores in advance information indicating a correspondence between an analysis result of the gait behavior and the perspective, and specifies the perspective by comparing the analysis result of the machine learning model with the information.

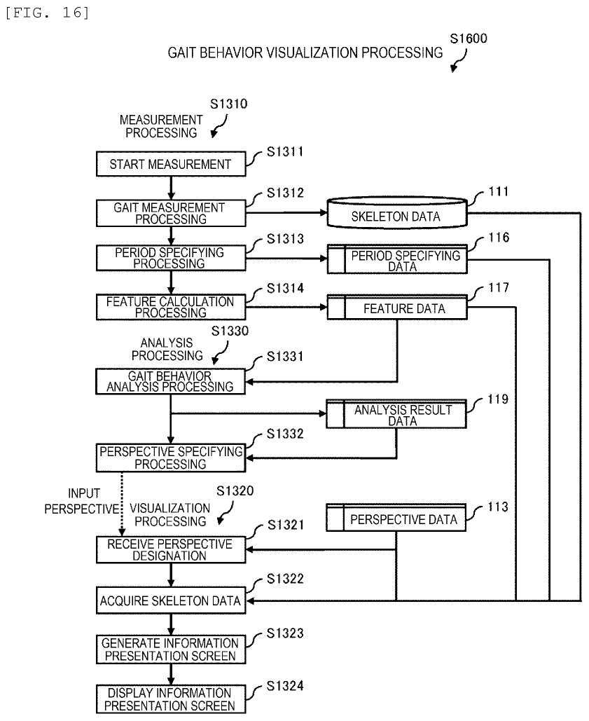

FIG. 16 is a flowchart showing processing performed by the gait behavior visualization device 100 of the second embodiment (hereinafter, referred to as “gait behavior visualization processing S1600”). As shown in FIG. 16, in the gait behavior visualization processing S1600 of the second embodiment, in addition to the measurement processing S1310 and the visualization processing S1320 described in the first embodiment, analysis processing S1330 is further executed. As shown in FIG. 16, the analysis processing S1330 includes gait behavior analysis processing S1331 and perspective specifying processing S1332.

In the gait behavior analysis processing S1331, the gait behavior analysis processing unit 150 analyzes the gait of the measurement subject by the machine learning model based on the feature data 117 generated by the feature calculation unit 135 to generate the analysis result data 191.

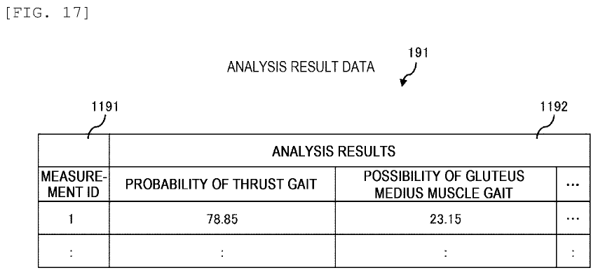

FIG. 17 shows an example of the analysis result data 191. The illustrated analysis result data 191 includes one or more entries (records) each including items of a measurement ID 1191 and an analysis result 1192. Among the above items, the measurement ID 1191 stores the above-described measurement ID (the measurement ID 1171 of the feature data 117 used for analysis). A value based on an output of the machine learning model is stored in the analysis result 1192. In the present example, a “probability of thrust gait” and a “probability of gluteus medius muscle gait” are illustrated as analysis results.

Returning to FIG. 16, in the perspective specifying processing S1332, the perspective specifying unit 152 specifies a perspective based on the analysis result data 191 and inputs the perspective to the perspective receiving unit 141 of the visualization processing unit 140. For example, in a case where the analysis result data 191 includes the content shown in FIG. 17, the perspective specifying unit 152 selects (specifies) a perspective suitable for displaying a gait behavior with a high probability, in the present example, since the “probability of thrust gait” is higher than the “probability of gluteus medius muscle gait”, the perspective specifying unit 152 selects (specifies) a perspective of an entry in which the perspective name 1131 is “thrust gait” when the perspective data 113 includes the content shown in FIG. 4, for example.

The visualization processing unit 140 generates an information presentation screen in the same manner as in the first embodiment based on the perspective input to the perspective receiving unit 141 by the perspective specifying unit 152, and transmits the generated information presentation screen to the user device 3.

As described above, the gait behavior visualization system 1 of the second embodiment analyzes the gait of the measurement subject by the machine learning model based on the feature calculated for the measurement subject, and automatically selects (specifies) a perspective based on the analysis result. Therefore, an information presentation screen on which the gait behavior is visualized from an appropriate perspective automatically selected by the machine learning model can be generated and the information presentation screen can be presented to the user.

### Third Embodiment

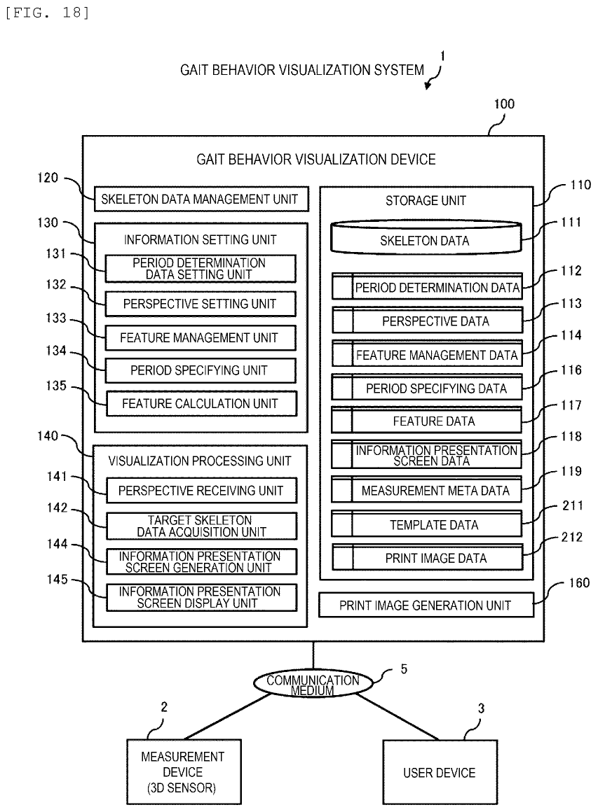

FIG. 18 shows a schematic configuration of the gait behavior visualization system 1 shown as a third embodiment. The gait behavior visualization device 100 in the gait behavior visualization system 1 of the third embodiment further includes a print image generation unit 160 in addition to functions of the gait behavior visualization device 100 of the first embodiment. The storage unit 110 of the gait behavior visualization system 1 of the third embodiment further stores template data 211 and print image data 212.

The print image generation unit 160 generates the print image data 212 (for example, file data in portable document format (PDF)) based on the information presentation screen data 118 generated by the visualization processing unit 140. The template data 211 is a template (layout form, and the like) of the print image data 212. The print image generation unit 160 generates the print image data 212 by applying information included in the information presentation screen data 118 to the template data 211. The print image generation unit 160 generates the print image data 212 using the skeleton data 111 as necessary.

The template data 211 is prepared in advance by, for example, an on-site user using application software for layout creation. For example, when a site is a medical site, the template data 211 is data designed for explanation of a gait behavior or the like to a client, and for example, when the site is a training gym, the template data 211 is data designed for instruction of training to a user.

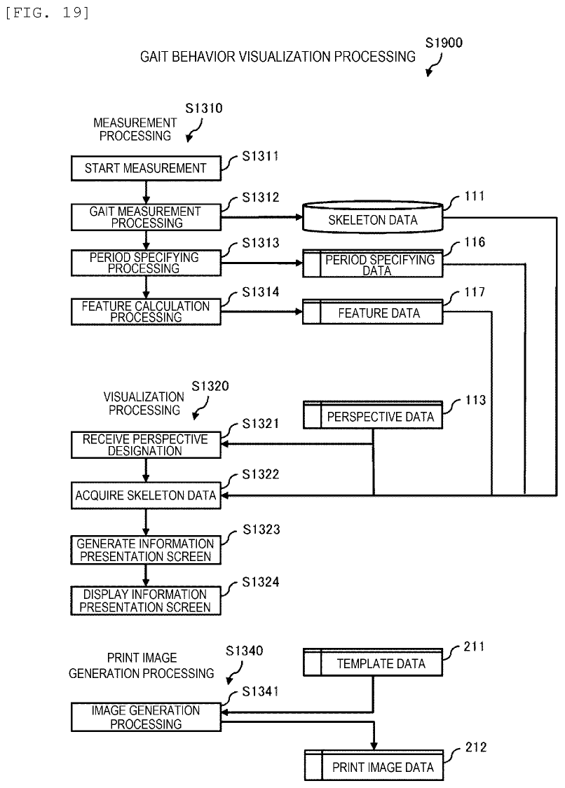

FIG. 19 is a flowchart showing processing performed by the gait behavior visualization device 100 of the third embodiment (hereinafter, referred to as “gait behavior visualization processing S1900”). As shown in FIG. 19, in the gait behavior visualization processing S1900 of the third embodiment, in addition to the measurement processing S1310 and the visualization processing S1320 described in the first embodiment, print image generation processing S1340 is further executed. As shown in FIG. 19, the print image generation processing S1340 includes image generation processing S1341.

In the image generation processing S1341, the print image generation unit 160 generates the print image data 212 by applying information included in the information presentation screen data 118 and information based on the skeleton data 111 to the template data 211.

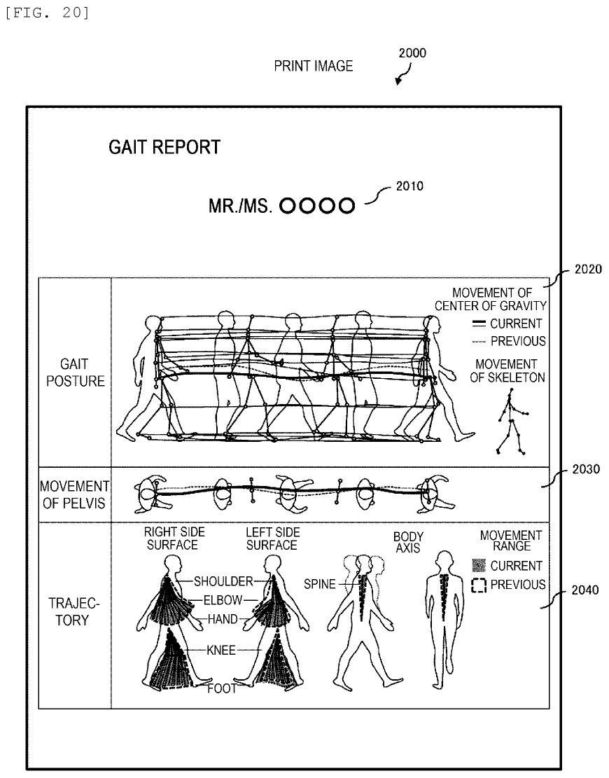

FIG. 20 shows an example of a print image generated based on the print image data 212. As shown in FIG. 20, in a print image 2000 to be illustrated, a name 2010 of the measurement subject (or the measurement subject ID), a trajectory (on a sagittal plane) 2020 of a center of gravity or a measurement point of the measurement subject for confirming a gait posture, a movement (on a horizontal plane) 2030 of a pelvis of the measurement subject, a trajectory (on a sagittal plane) 2040 of a line segment connecting sites, and the like are described.

For example, the user can print (print out) the print image 2000 on a paper medium or the like excellent in portability and convenience by performing a print instruction operation on the user device 3.

Although the embodiments of the invention have been described above, the invention is not limited to the embodiments, and it is needless to say that various modifications can be made without departing from the gist of the present invention. For example, the embodiments described above have been described in detail to facilitate understanding of the invention, and the invention is not necessarily limited to those including all the configurations described above. In addition, another configuration can be added to, deleted from, or replaced with a part of a configuration of each embodiment.

In the above embodiments, a case where the gait behavior of a person is visualized has been described as an example, but the gait behavior visualization system 1 can be applied to a case where a gait behavior of a person other than a gait is visualized.

In the above embodiments, a case where the measurement subject is a person has been described as an example, but the gait behavior visualization system 1 can also be applied to a case where the measurement subject is one other than a person such as an animal.

A part or all of the configurations, function units, processing units, processing methods, and the like described above may be implemented by hardware by, for example, designing with an integrated circuit. In addition, the configurations, functions, and the like described above may be implemented by software by a processor interpreting and executing a program for implementing each function. Information such as a program, a table, and a file for implementing each function can be stored in a recording device such as a memory, a hard disk, and a solid state drive (SSD), or in a recording medium such as an IC card, an SD card, and a DVD.

In the drawings, control lines and information lines that are considered necessary for explanation are shown, and not all control lines and information lines on implementation are necessarily shown. For example, it may be considered that almost all configurations are actually interconnected.

Arrangements of the various functional units, various processing units, and various databases of the information processing system described above are merely examples. The arrangements of the various functional units, various processing units, and various databases may be changed to optimal arrangements from the viewpoint of performance, processing efficiency, communication efficiency, and the like of hardware and software provided in the gait behavior visualization system 1.

In addition, the configuration (schema, and the like) of the above-described various pieces of data and various databases may be flexibly changed from the viewpoint of efficient use of resources, improvement in processing efficiency, improvement in access efficiency, improvement in search efficiency, and the like.

### Reference Signs List

1: gait behavior visualization system
2: measurement device
3: user device
5: communication medium
100: gait behavior visualization device
111: skeleton data
112: period determination data
113: perspective data
114: feature management data
116: period specifying data
117: feature Data
118: information presentation screen data
191: analysis result data
120: skeleton data management unit
130: information setting unit
131: period determination data setting unit
132: perspective setting unit
133: feature management unit
134: period specifying unit
135: feature calculation unit
140: visualization processing unit
141: perspective receiving unit
142: target skeleton data acquisition unit
144: information presentation screen generation unit
145: information presentation screen display unit
150: gait behavior analysis processing unit
151: gait behavior analysis unit
152: perspective specifying unit
160: print image generation unit
211: template data
212: print image data
1410: information presentation screen
S1300: gait behavior visualization processing
S1600: gait behavior visualization processing
S1900: gait behavior visualization processing
2000: print image

1. A gait behavior visualization method comprising:
a step of storing, using an information processing device including a processor and a memory, skeleton data, which is time-series data indicating trajectories of a plurality of sites set for a measurement subject in a three-dimensional space, for a gait of the measurement subject, period determination data, which is information used for specifying a time corresponding to each of periods of one gait, for the skeleton data, and a perspective defined by designating the period, the site, and a displaying method for the trajectory;
a step of specifying, using the information processing device, a time corresponding to the period for the skeleton data by comparing the skeleton data and the period determination data; and
a step of generating, using the information processing device based on the skeleton data for the period designated in the perspective, a screen on which a trajectory of the site designated in the perspective is presented according to the displaying method designated in the perspective.

2. The gait behavior visualization method according to claim 1, wherein
the information processing device further executes a step of storing a feature calculated based on the skeleton data,
the perspective is defined by further designating the feature, and
the information processing device further executes a step of generating the screen on which information based on the feature designated in the perspective is presented together with the trajectory of the site.

3. The gait behavior visualization method according to claim 2, wherein
the information based on the feature is a graph showing a temporal change of the feature.

4. The gait behavior visualization method according to claim 1, wherein
the information processing device further executes a step of generating the screen on which a trajectory of a line segment connecting the plurality of sites designated in the perspective is presented together with the trajectories of the plurality of sites.

5. The gait behavior visualization method according to claim 1, wherein
the perspective is defined by further designating a reference point which is a reference position when displaying the trajectory, and
the information processing device further executes a step of generating, based on the skeleton data for the period designated in the perspective, the screen on which the trajectory of the site designated in the perspective is shown relative to the reference point designated in the perspective.

6. The gait behavior visualization method according to claim 1, wherein
the information processing device further executes
a step of storing a plurality of the perspectives having different designated contents, and
a step of generating the screen on which the trajectories of the sites are simultaneously displayed based on each of the perspectives.

7. The gait behavior visualization method according to claim 1, wherein
the information processing device further executes a step of generating the screen on which the trajectories of the sites based on each of a plurality of the measurement subjects are simultaneously displayed.

8. The gait behavior visualization method according to claim 1, wherein
the information processing device further executes
a step of storing a feature calculated based on the skeleton data,
a step of analyzing a gait behavior of the measurement subject by inputting the feature to a machine learning model, and
a step of generating, based on the skeleton data for the period designated in the perspective specified based on the gait behavior obtained by analysis, a screen on which the trajectory of the site designated in the perspective is presented according to the displaying method designated in the perspective.

9. The gait behavior visualization method according to claim 1, wherein
the information processing device further executes
a step of storing template data used to generate a print image, and
a step of generating a print image capable of being printed on a paper medium by applying a content of the screen to the template data.

10. The gait behavior visualization method according to claim 1, wherein
the information processing device further includes a user interface configured to receive a designation of the perspective, and
the information processing device further executes
a step of receiving a designation of the perspective from a user via the user interface, and
a step of generating, based on the skeleton data for the period designated in the received perspective, a screen on which the trajectory of the site designated in the perspective is presented according to the displaying method designated in the perspective.

11. The gait behavior visualization method according to claim 1, wherein
the information processing device further includes a user interface configured to receive a setting of the perspective from a user, and
the information processing device further executes a step of receiving a setting of a content of the perspective via the user interface.

12. A program for causing an information processing device including a processor and a memory to implement:
a function of storing skeleton data, which is time-series data indicating trajectories of a plurality of sites set for a measurement subject in a three-dimensional space, for a gait of the measurement subject, period determination data, which is information used for specifying a time corresponding to each of periods of one gait, for the skeleton data, and a perspective defined by designating the period, the site, and a displaying method for the trajectory;
a function of specifying a time corresponding to the period for the skeleton data by comparing the skeleton data and the period determination data; and
a function of generating, based on the skeleton data for the period designated in the perspective, a screen on which the trajectory of the site designated in the perspective is presented according to the displaying method designated in the perspective.

13. An information processing device comprising:
a processor; and
a memory, wherein
the device
stores skeleton data, which is time-series data indicating trajectories of a plurality of sites set for a measurement subject in a three-dimensional space, for a gait of the measurement subject, period determination data, which is information used for specifying a time corresponding to each of periods of one gait, for the skeleton data, and a perspective defined by designating the period, the site, and a displaying method for the trajectory,
specifies a time corresponding to the period for the skeleton data by comparing the skeleton data and the period determination data, and
generates, based on the skeleton data in the period designated in the perspective, a screen on which the trajectory of the site designated in the perspective is presented according to the displaying method designated in the perspective.

14. The information processing device according to claim 13, wherein
the device
stores a feature calculated based on the skeleton data,
analyzes a gait behavior of the measurement subject by inputting the feature to a machine learning model, and
generates, based on the skeleton data for the period designated in the perspective specified based on the gait behavior obtained by analysis, a screen on which the trajectory of the site designated in the perspective is presented according to the displaying method designated in the perspective.

15. The information processing device according to claim 13, wherein
the device
stores template data used to generate a print image, and
generates a print image capable of being printed on a paper medium by applying a content of the screen to the template data.
